(19) United States
(12) Patent Application Publication
(10) Pub. No.: US 2020/0188477 A1
(43) Pub. Date: Jun. 18, 2020

(54) A MEDICAMENT FOR THE TREATMENT OF DISEASES BY BIOFILM FORMING MICROORGANISMS

(71) Applicant: Indian institute of Technology Delhi, New Delhi (IN)

(72) Inventors: Seyed Ehtesham Hasnain, New Delhi (IN); Nasreen Zafar Ehtesham, New Delhi (IN); Deeksha Tripathi, New Delhi (IN); Sonam Grover, New Delhi (IN); Ashutosh Kumar, New Delhi (IN); Anwar Alam, New Delhi (IN); Saurabh Pandey, New Delhi (IN)

(21) Appl. No.: 16/607,061

(22) PCT Filed: Apr. 20, 2018
(86) PCT No.: PCT/IN2018/050238

§ 371 Date: Oct. 21, 2019

(30) Foreign Application Priority Data
Apr. 21, 2017 (IN) 201711014310

(51) Int. Cl. A61K 38/13; A61K 31/133; A61K 31/4409; A61K 31/496; A61K 31/702; A61K 31/7036; A61K 31/7048; A61K 33/26; A61K 38/46

(52) U.S. Cl. CPC A61K 38/13; A61K 31/133; A61K 31/4409; A61K 31/496; A61K 38/465; A61K 31/7036; A61K 31/7048; A61K 33/26; A61K 31/702

(57) ABSTRACT

The present disclosure discloses a medicament comprising a drug selected from a group consisting of acarbose, cyclosporine A, its pharmaceutically acceptable salts thereof, and combinations thereof, for use in treatment of diseases caused by biofilm forming microorganisms. Also, provided herein are methods of treating diseases caused by biofilm forming microorganisms.

### FIELD OF INVENTION

The present disclosure, in general relates to the field of disease management, and in particular, relates to a medicament including a drug selected from the group consisting of acarbose, Cyclosporine A, its pharmaceutically acceptable salts thereof, and combinations thereof for the treatment of diseases caused by biofilm forming microorganisms.

### BACKGROUND OF THE INVENTION

Mycobacterium tuberculosis is an obligate pathogenic bacterial species and the causative agent of tuberculosis. Primarily a pathogen of the mammalian respiratory system, it infects the lungs. M. tuberculosis divides every 15-20 hours, which is extremely slow compared with other bacteria. When in the lungs, M. tuberculosis is taken up by alveolar macrophages, but they are unable to digest and eradicate the bacterium. M. tuberculosis and Mycobacterium smegmatis have a strong propensity to grow in organized multicellular structures called biofilms. M. tuberculosis shares several characteristics with organisms that produce biofilms during infection. For example, infections caused by M. tuberculosis are highly persistent, show resistance to antimicrobials, and exhibit the ability to modulate the host immune system.

M. tuberculosis has two peptidyl-prolylisomerase (Ppiases) PpiA and PpiB, popularly known as cyclophilin. M. tuberculosis PpiB is a surface localized glycoprotein. The M. tuberculosis PpiB gene, encoding PpiB protein, is an essential gene, which when knocked out does not allow the bacterium to survive. Expression of M. tuberculosis PpiB gene in M. smegmatis increases bacterial survivability inside macrophages (Pandey S (2016) Mycobacterium Tuberculosis peptidyl-prolylisomerase: biochemical, immunological and functional characterization. PhD Thesis—submitted to University of Hyderabad, India. It has been reported that Mycobacterial protein, the polyketide Pks1 contributes to biofilm formation in M. tuberculosis (Pang et al, J. Bac. 2012, 194(3):715-21) but inhibitor against this protein is not known. It has also been reported that a chemical, TCA1 reduces the biofilm formation by M. tuberculosis (Wang et al, PNAS, USA, 2013; 110(27): E2510-7) but its target has not been studied.

The most frequently used diagnostic methods for tuberculosis are the tuberculin skin test, acid-fast stain, and chest radiographs. Treatment of tuberculosis frequently involves multi drug therapy (MDT) treatment. This involves taking a combination of four drugs for a period of 6-9 months, often causing toxicity to the patients. The prolonged treatment is due to the ability of this slow growing bacterium to form the biofilm which makes the bacterium drug tolerant.

The development of drug resistance in this bacterium has led to the development of M. tuberculosis variants which show multi-drug resistance (MDR). This is a major issue in the treatment of the disease and there is now a need to identify alternative drugs for the treatment of tuberculosis.

### SUMMARY OF THE INVENTION

In an aspect of the present disclosure, there is provided a medicament comprising a drug selected from a group consisting of acarbose, cyclosporine A, its pharmaceutically acceptable salts thereof, and combinations thereof, for use in treatment of diseases caused by biofilm forming microorganisms.

In an aspect of the present disclosure, there is provided a method of treatment of diseases caused by biofilm forming microorganisms, said method comprising:

(a) obtaining a drug selected from a group consisting of acarbose and its pharmaceutically acceptable salts; and
(b) administering a therapeutically relevant amount of acarbose.

In an aspect of the present disclosure, there is provided a method of treatment of diseases caused by biofilm forming microorganisms, said method comprising:

(a) obtaining a drug selected from a group consisting of Cyclosporine A and its pharmaceutically acceptable salts; and
(b) administering a therapeutically relevant amount of Cyclosporine A.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE ACCOMPANYING DRAWINGS

The following figures form part of the present specification and are included to further illustrate aspects of the present disclosure. The disclosure may be better understood by reference to the figures in combination with the detailed description of the specific embodiments presented herein.

FIG. 1 depicts the enhanced biofilm and pellicle formation in recombinant M. smegmatis expressing M. tuberculosis PpiB protein, in accordance with an embodiment of the present disclosure.

FIG. 2 depicts the effect of Cyclosporine A on the viability of M. smegmatis using the alamar blue assay, in accordance with an embodiment of the present disclosure.

FIG. 3 depicts the effect of acarbose on the viability of M. smegmatis using the alamar blue assay, in accordance with an embodiment of the present disclosure.

FIG. 4 depicts the effect of Cyclosporine A on biofilm formation in M. smegmatis expressing M. tuberculosis PpiB, using the crystal violet assay, in accordance with an embodiment of the present disclosure.

FIG. 5 depicts the effect of acarbose on biofilm formation in M. smegmatis expressing M. tuberculosis PpiB, using the crystal violet assay, in accordance with an embodiment of the present disclosure.

FIG. 6 depicts the effect of isoniazide on survival of M. smegmatis expressing M. tuberculosis PpiB in the presence and absence of Cyclosporine A, in accordance with an embodiment of the present disclosure.

FIG. 7 depicts effect of ethambutol on the survival of M. smegmatis expressing M. tuberculosis PpiB in the presence and absence of Cyclosporine A, in accordance with an embodiment of the present disclosure.

FIG. 8 depicts the effect of isoniazide on survival of M. smegmatis expressing M. tuberculosis PpiB, in the presence and absence of acarbose, in accordance with an embodiment of the present disclosure.

FIG. 9 depicts an RMSD (Root Mean Square Deviation) plot of PpiB apo-structure molecular dynamics (MD) trajectory, in accordance with an embodiment of the present disclosure.

FIG. 10 depicts ribbon representation showing superimposition of the Pre-MD PpiB (shown in blue) and Post-MD PpiB structure obtained from the 40 ns molecular dynamic simulations (shown in golden-yellow), in accordance with an embodiment of the present disclosure.

FIG. 11 depicts the interactions of PpiB with acarbose, in accordance with an embodiment of the present disclosure.

FIG. 12 depicts the RMSD plot of PpiB docked complexes of acarbose MD trajectory, in accordance with an embodiment of the present disclosure.

FIG. 13 depicts the interactions of PpiB with acarbose after 10 ns molecular dynamics simulation, in accordance with an embodiment of the present disclosure.

FIG. 14 depicts suppressive effect of Cyclosporine A on biofilm formation in Mycobacterium tuberculosis H37Rv strains, in accordance with an embodiment of the present disclosure.

FIGS. 15A and 15B depicts multiple interactions between cyclosporine A and M. tb PpiB, in accordance with an embodiment of the present disclosure.

FIG. 16 depicts protein homology of M. tb PpiB (Rv2582) with proteins from other biofilm forming bacteria, in accordance with an embodiment of the present disclosure.

FIG. 17 A depicts homology of M. tb PpiA or PpiB with E. coli Ppiase, and FIG. 17 B depicts homology of M. tb Ppiase with trigger factor proteins (RopA) expressed in S. mutans, in accordance with an embodiment of the present disclosure.

FIG. 18 A depicts SPR analysis of cyclosporine A interaction with M. tb PpiB, and FIG. 18 B depicts SPR analysis of acarbose interaction with M. tb PpiB, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any or all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “biofilm forming organisms” refers to microorganisms in general, and bacteria in particular, capable of forming biofilms. The ones contemplated by the present invention are Mycobacterium smegmatis, Mycobacterium tuberculosis, drug resistant M. tuberculosis, multidrug resistant M. tuberculosis, extensively drug resistant M. tuberculosis or totally drug resistant M. tuberculosis, variants and combinations thereof. Apart from the mentioned microorganisms, the present invention also includes Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

The terms “multidrug resistant M. tuberculosis, extensively drug resistant M. tuberculosis or totally drug resistant M. tuberculosis” refer to variants of M. tuberculosis that are resistant to most commonly used drugs employed against the bacterium, including rifampicin, isoniazid and any fluoroquinolone.

The term “acarbose” refers to a drug used for the management of type 2 Diabetes mellitus. It delays the release of glucose from complex carbohydrates and disaccharides by acting as an inhibitor of alpha-glucosidases in the intestine and thereby attenuating postprandial increments in glucose as well as insulin in the blood.

The term “cyclosporine A” refers to an immunosuppressant drug widely used in organ transplantation to prevent rejection. It reduces the activity of the immune system by interfering with the activity and growth of T cells and is a known inhibitor of cyclophilins.

The term therapeutically “relevant amount of drug” as used in the description refers to the amount of drug that is to be provided to a subject, such that, the drug exhibits the function as disclosed on the present invention, i.e. treatment of diseases caused by biofilm forming microorganisms.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally-equivalent products, compositions, and methods are clearly within the scope of the disclosure, as described herein.

The present disclosure relates to a medicament comprising a drug selected from the group consisting of acarbose, cyclosporine A, its pharmaceutically acceptable salts thereof, for use in the treatment of diseases caused by biofilm forming microorganisms.

In an embodiment of the present disclosure, there is provided a medicament comprising a drug selected from a group consisting of acarbosea, cyclosporine A, its pharmaceutically acceptable salts thereof; and combinations thereof, for use in treatment of diseases caused by biofilm forming microorganisms.

In an embodiment of the present disclosure, there is provided a medicament comprising cyclosporine A for use in treatment of diseases caused by biofilm forming microorganisms.

In an embodiment of the present disclosure, there is provided a medicament comprising pharmaceutically acceptable salt of cyclosporine A for use in treatment of diseases caused by biofilm forming microorganisms.

In an embodiment of the present disclosure, there is provided a medicament comprising acarbose for use in treatment of diseases caused by biofilm forming microorganisms.

In an embodiment of the present disclosure, there is provided a medicament comprising pharmaceutically acceptable salt of acarbose for use in treatment of diseases caused by biofilm forming microorganisms.

In an embodiment of the present disclosure, there is provided a medicament comprising a drug selected from group consisting of acarbose, cyclosporine A, its pharmaceutically acceptable salts thereof, and combinations thereof; and at least one compound for use in treatment of diseases caused by biofilm forming microorganisms.

In an embodiment of the present disclosure, there is provided a medicament comprising cyclosporine A; and at least one compound for use in treatment of diseases caused by biofilm forming microorganisms.

In an embodiment of the present disclosure, there is provided a medicament comprising pharmaceutically acceptable salt of cyclosporine A; and at least one compound for use in treatment of diseases caused by biofilm forming microorganisms.

In an embodiment of the present disclosure, there is provided a medicament comprising acarbose; and at least one compound for use in treatment of diseases caused by biofilm forming microorganisms.

In an embodiment of the present disclosure, there is provided a medicament comprising pharmaceutically acceptable salt of acarbose; and at least one compound for use in treatment of diseases caused by biofilm forming microorganisms.

In an embodiment of the present disclosure, there is provided a medicament comprising a drug selected from a group consisting of acarbose, cyclosporine A, its pharmaceutically acceptable salts thereof; and combinations thereof, for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising cyclosporine A for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of cyclosporine A for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising acarbose for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of acarbose for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising a drug selected from a group consisting of acarbose, cyclosporine A, its pharmaceutically acceptable salts thereof, and combinations thereof; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising cyclosporine A; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of cyclosporine A; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising acarbose; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of acarbose; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising a drug selected from a group consisting of acarbose, cyclosporine A, its pharmaceutically acceptable salts thereof, and combinations thereof for use in treatment of diseases caused by a biofilm forming microorganism: Mycobacterium tuberculosis.

In an embodiment of the present disclosure, there is provided a medicament comprising a drug selected from a group consisting of acarbose, cyclosporine A, its pharmaceutically acceptable salts thereof, and combinations thereof; and at least one compound for use in treatment of diseases caused by a biofilm forming microorganism: Mycobacterium tuberculosis.

In an embodiment of the present disclosure, there is provided a medicament comprising cyclosporine A for use in treatment of diseases caused by a biofilm forming microorganism: Mycobacterium tuberculosis.

In an embodiment of the present disclosure, there is provided a medicament comprising pharmaceutically acceptable salt of cyclosporine A for use in treatment of diseases caused by a biofilm forming microorganism: Mycobacterium tuberculosis.

In an embodiment of the present disclosure, there is provided a medicament comprising cyclosporine A; and at least one compound for use in treatment of diseases caused by a biofilm forming microorganism: Mycobacterium tuberculosis.

In an embodiment of the present disclosure, there is provided a medicament comprising pharmaceutically acceptable salt of cyclosporine A; and at least one compound for use in treatment of diseases caused by a biofilm forming microorganism: Mycobacterium tuberculosis.

In an embodiment of the present disclosure, there is provided a medicament comprising acarbose for use in treatment of diseases caused by a biofilm forming microorganism: Mycobacterium tuberculosis.

In an embodiment of the present disclosure, there is provided a medicament comprising pharmaceutically acceptable salt of acarbose for use in treatment of diseases caused by a biofilm forming microorganism: Mycobacterium tuberculosis.

In an embodiment of the present disclosure, there is provided a medicament comprising acarbose; and at least one compound for use in treatment of diseases caused by a biofilm forming microorganism: Mycobacterium tuberculosis.

In an embodiment of the present disclosure, there is provided a medicament comprising pharmaceutically acceptable salt of acarbose; and at least one compound for use in treatment of diseases caused by biofilm forming microorganism: Mycobacterium tuberculosis.

In an embodiment of the present disclosure, there is provided a medicament comprising a drug selected from a group consisting of acarbose, cyclosporine A, its pharmaceutically acceptable salts thereof; and combinations thereof for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising cyclosporine A for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising pharmaceutically acceptable salt of cyclosporine A for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising acarbose for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising pharmaceutically acceptable salt of acarbose for use in the treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising a drug selected from a group consisting of acarbose, cyclosporine A, its pharmaceutically acceptable salts thereof, and combinations thereof; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising cyclosporine A; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising pharmaceutically acceptable salt of cyclosporine A; and at least one compound for use in treatment of diseases caused by a biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising acarbose; and at least one compound for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising pharmaceutically acceptable salt of acarbose; and at least one compound for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, and periodontitis.

In an embodiment of the present disclosure, there is provided a medicament comprising a drug selected from a group consisting of acarbose, cyclosporine A, its pharmaceutically acceptable salts thereof, and combinations thereof for use in treatment of tuberculosis.

In an embodiment of the present disclosure, there is provided a medicament comprising a drug selected from the group consisting of acarbose, cyclosporine A, its pharmaceutically acceptable salts thereof, and combinations thereof for use in treatment of dental caries.

In an embodiment of the present disclosure, there is provided a medicament comprising a drug selected from a group consisting of acarbose, cyclosporine A, its pharmaceutically acceptable salts thereof, and combinations thereof for use in treatment of periodontitis.

In an embodiment of the present disclosure, there is provided a medicament comprising cyclosporine A for use in treatment of tuberculosis.

In an embodiment of the present disclosure, there is provided a medicament comprising cyclosporine A for use in treatment of dental caries.

In an embodiment of the present disclosure, there is provided a medicament comprising cyclosporine A for use in treatment of periodontitis.

In an embodiment of the present disclosure, there is provided a medicament comprising pharmaceutically acceptable salt of cyclosporine A for use in treatment of tuberculosis.

In an embodiment of the present disclosure, there is provided a medicament comprising pharmaceutically acceptable salt of cyclosporine A for use in treatment of dental caries.

In an embodiment of the present disclosure, there is provided a medicament comprising pharmaceutically acceptable salt of cyclosporine A for use in treatment of periodontitis.

In an embodiment of the present disclosure, there is provided a medicament comprising cyclosporine A; and at least one compound for use in treatment of tuberculosis.

In an embodiment of the present disclosure, there is provided a medicament comprising cyclosporine A; and at least one compound for use in treatment of dental caries.

In an embodiment of the present disclosure, there is provided a medicament comprising cyclosporine A; and at least one compound for use in treatment of periodontitis.

In an embodiment of the present disclosure, there is provided a medicament comprising pharmaceutically acceptable salt of cyclosporine A; and at least one compound for use in treatment of tuberculosis.

In an embodiment of the present disclosure, there is provided a medicament comprising pharmaceutically acceptable salt of cyclosporine A; and at least one compound for use in treatment of dental caries.

In an embodiment of the present disclosure, there is provided a medicament comprising pharmaceutically acceptable salt of cyclosporine A; and at least one compound for use in treatment of periodontitis.

In an embodiment of the present disclosure, there is provided a medicament comprising acarbose for use in treatment of tuberculosis.

In an embodiment of the present disclosure, there is provided a medicament comprising acarbose for use in treatment of dental caries.

In an embodiment of the present disclosure, there is provided a medicament comprising acarbose for use in treatment of periodontitis.

In an embodiment of the present disclosure, there is provided a medicament comprising pharmaceutically acceptable salt of acarbose for use in treatment of tuberculosis.

In an embodiment of the present disclosure, there is provided a medicament comprising pharmaceutically acceptable salt of acarbose for use in treatment of dental caries.

In an embodiment of the present disclosure, there is provided a medicament comprising pharmaceutically acceptable salt of acarbose for use in treatment of periodontitis.

In an embodiment of the present disclosure, there is provided a medicament comprising acarbose; and at least one compound for use in treatment of tuberculosis.

In an embodiment of the present disclosure, there is provided a medicament comprising acarbose; and at least one compound for use in treatment of dental caries.

In an embodiment of the present disclosure, there is provided a medicament comprising acarbose; and at least one compound for use in treatment of periodontitis.

In an embodiment of the present disclosure, there is provided a medicament comprising pharmaceutically acceptable salt of acarbose; and at least one compound for use in treatment of tuberculosis.

In an embodiment of the present disclosure, there is provided a medicament comprising acarbose; and at least one compound for use in treatment of dental caries.

In an embodiment of the present disclosure, there is provided a medicament comprising acarbose; and at least one compound for use in treatment of periodontitis.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism, wherein said diseases are selected from a group consisting of tuberculosis, dental caries, and periodontitis.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, periodontitis.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis.

In an embodiment of the present disclosure, there is provided a medicament comprising cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism.

In an embodiment of the present disclosure, there is provided a medicament comprising cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis.

In an embodiment of the present disclosure, there is provided a medicament comprising pharmaceutically acceptable salt of cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism.

In an embodiment of the present disclosure, there is provided a medicament comprising pharmaceutically acceptable salt of cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of cyclosporine A; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis.

In an embodiment of the present disclosure, there is provided a medicament comprising acarbose; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism.

In an embodiment of the present disclosure, there is provided a medicament comprising acarbose; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising acarbose; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis.

In an embodiment of the present disclosure, there is provided a medicament comprising pharmaceutically acceptable salt of acarbose; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of acarbose; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis.

In an embodiment of the present disclosure, there is provided a medicament comprising acarbose and isoniazide for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of acarbose and isoniazide for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a medicament comprising pharmaceutically acceptable salt of acarbose and isoniazide for treatment of diseases caused by biofilm forming microorganism: M. tuberculosis.

In an embodiment of the present disclosure, there is provided a medicament comprising acarbose and ethambutol for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, and combinations thereof.

In an embodiment of the present disclosure, there is provided a medicament comprising acarbose and ethambutol for treatment of diseases caused by biofilm forming microorganism: M. tuberculosis.

In an embodiment of the present disclosure, there is provided a medicament comprising pharmaceutically acceptable salt of acarbose and ethambutol for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, and combinations thereof.

In an embodiment of the present disclosure, there is provided a medicament comprising pharmaceutically acceptable salt of acarbose and ethambutol for treatment of diseases caused by biofilm forming microorganism: M. tuberculosis.

In an embodiment of the present disclosure, there is provided a medicament comprising cyclosporine A and isoniazide for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, and combinations thereof.

In an embodiment of the present disclosure, there is provided a medicament comprising cyclosporine A and isoniazide for treatment of diseases caused by biofilm forming organism: M. tuberculosis.

In an embodiment of the present disclosure, there is provided a medicament comprising pharmaceutically acceptable salt of cyclosporine A and isoniazide for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis, totally drug resistant M. tuberculosis, and combinations thereof.

In an embodiment of the present disclosure, there is provided a medicament comprising pharmaceutically acceptable salt of cyclosporine A and isoniazide for treatment of diseases caused by biofilm forming microorganism: M. tuberculosis.

In an embodiment of the present disclosure, there is provided a medicament comprising cyclosporine A and ethambutol for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising cyclosporine A and ethambutol for treatment of diseases caused by biofilm forming microorganism: M. tuberculosis.

In an embodiment of the present disclosure, there is provided a medicament comprising pharmaceutically acceptable salt of cyclosporine A and ethambutol for treatment of diseases caused by biofilm forming microorganism selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising pharmaceutically acceptable salt of cyclosporine A and ethambutol for treatment of diseases caused by biofilm forming microorganism: M. tuberculosis.

In an embodiment of the present disclosure, there is provided a medicament comprising acarbose to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose to inhibit the growth or activity or both, of biofilm forming microorganisms: M. tuberculosis.

In an embodiment of the present disclosure, there is provided a medicament comprising a pharmaceutically acceptable salt of acarbose to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt of acarbose to inhibit the growth or activity or both, of biofilm forming microorganisms: M. tuberculosis.

In an embodiment of the present disclosure, there is provided a medicament comprising cyclosporine A to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising cyclosporine A to inhibit the growth or activity or both, of biofilm forming microorganisms: M. tuberculosis.

In an embodiment of the present disclosure, there is provided a medicament comprising a pharmaceutically acceptable salt of cyclosporine A to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt of cyclosporine A to inhibit the growth or activity or both, of biofilm forming microorganisms: M. tuberculosis.

In an embodiment of the present disclosure, there is provided a medicament comprising acarbose and isoniazide, to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose and isoniazide, to inhibit the growth or activity or both, of biofilm forming microorganisms M. tuberculosis.

In an embodiment of the present disclosure, there is provided a medicament comprising a pharmaceutically acceptable salt of acarbose and isoniazide, to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt of acarbose and isoniazide, to inhibit the growth or activity or both, of biofilm forming microorganisms: M. tuberculosis.

In an embodiment of the present disclosure, there is provided a medicament comprising acarbose and ethambutol to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose and ethambutol to inhibit the growth or activity or both, of biofilm forming microorganisms: M. tuberculosis.

In an embodiment of the present disclosure, there is provided a medicament comprising a pharmaceutically acceptable salt of acarbose and ethambutol to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt of acarbose and ethambutol to inhibit the growth or activity or both, of biofilm forming microorganisms: M. tuberculosis.

In an embodiment of the present disclosure, there is provided a medicament comprising cyclosporine A and isoniazide to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising cyclosporine A and isoniazide to inhibit the growth or activity or both, of biofilm forming microorganisms: M. tuberculosis.

In an embodiment of the present disclosure, there is provided a medicament comprising a pharmaceutically acceptable salt of cyclosporine A and isoniazide, to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt of cyclosporine A and isoniazide, to inhibit the growth or activity or both, of biofilm forming microorganisms: M. tuberculosis.

In an embodiment of the present disclosure, there is provided a medicament comprising cyclosporine A and ethambutol to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising cyclosporine A and ethambutol to inhibit the growth or activity or both, of biofilm forming microorganisms: M. tuberculosis.

In an embodiment of the present disclosure, there is provided a medicament comprising a pharmaceutically acceptable salt cyclosporine A and ethambutol to inhibit the growth or activity or both, of biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising a pharmaceutically acceptable salt cyclosporine A and ethambutol to inhibit the growth or activity or both, of biofilm forming microorganisms: M. tuberculosis.

In an embodiment of the present disclosure, there is provided a medicament comprising acarbose, and at least a single dose of acarbose is administered for treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose, and at least a single dose of acarbose is administered for treatment of diseases caused by biofilm forming microorganisms: M. tuberculosis.

In an embodiment of the present disclosure, there is provided a medicament comprising cyclosporine A, and at least a single dose of cyclosporine A is administered for treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising cyclosporine A, and at least a single dose of cyclosporine A is administered for treatment of diseases caused by biofilm forming microorganisms: M. tuberculosis.

In an embodiment of the present disclosure, there is provided a medicament comprising acarbose and isoniazide, and at least a single dose of acarbose and isoniazide is administered for treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose and isonizide, and at least a single dose of acarbose and isoniazide is administered for treatment of diseases caused by biofilm forming microorganisms: M. tuberculosis.

In an embodiment of the present disclosure, there is provided a medicament comprising pharmaceutically acceptable salt of acarbose, and isonizide, and at least a single dose of a pharmaceutically acceptable salt of acarbose and isoniazide is administered for treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising pharmaceutically acceptable salt of acarbose, and isoniazide, and at least a single dose of a pharmaceutically acceptable salt of acarbose and isoniazide is administered for the treatment of diseases caused by biofilm forming microorganisms: M. tuberculosis.

In an embodiment of the present disclosure, there is provided a medicament comprising acarbose and athambutol, and at least a single dose of acarbose and ethambutol, is administered for the treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising acarbose and ethambutol, and at least a single dose of acarbose and ethambutol, is administered for treatment of diseases caused by biofilm forming microorganisms: M. tuberculosis.

In an embodiment of the present disclosure, there is provided a medicament comprising pharmaceutically acceptable salt of acarbose and ethambutol, and at least a single dose of a pharmaceutically acceptable salt of acarbose and ethambutol, is administered for the treatment of diseases caused by biofilm forming microorganisms selected from a group consisting of Mycobacterium smegmatis, Mycobacterium tuberculosis, drug resistant M. tuberculosis, multidrug resistant M. tuberculosis, extensively drug resistant M. tuberculosis totally drug resistant M. tuberculosis, and combinations thereof.

In an embodiment of the present disclosure, there is provided a medicament comprising pharmaceutically acceptable salt of acarbose and ethambutol, and, at least a single dose of a pharmaceutically acceptable salt of acarbose and ethambutol, is administered for the treatment of diseases caused by biofilm forming microorganisms: M. tuberculosis.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism, wherein said compound is administered, before, concurrently, or after administration of the drug.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said compound is administered, before, concurrently, or after administration of the drug.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism, wherein said diseases are selected from a group consisting of tuberculosis, dental caries, and periodontitis, and wherein said compound is administered, before, concurrently, or after administration of the drug.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof, wherein said diseases are selected from a group consisting of tuberculosis, dental caries, periodontitis, and wherein said compound is administered, before, concurrently, or after administration of the drug.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis, and wherein said compound is administered, before, concurrently, or after administration of the drug.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism, wherein said compound is administered, before, concurrently, or after administration of the drug, and wherein said administration of said compound, and the drug can be in the form of a single dose or multiple doses.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, and combinations thereof, wherein said compound is administered, before, concurrently, or after administration of the drug, and wherein said administration of said compound, and the drug can be in the form of a single dose or multiple doses.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism, wherein said diseases are selected from a group consisting of tuberculosis, dental caries, and periodontitis, and wherein said compound is administered, before, concurrently, or after administration of the drug, and wherein said administration of said compound, and the drug can be in the form of a single dose or multiple doses.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism selected from a group consisting of Mycobacterium smegmatis, Mycobacterium tuberculosis, multidrug resistant M. tuberculosis, extensively drug resistant M. tuberculosis, totally drug resistant variant of M. tuberculosis, and combinations thereof, wherein said diseases are selected from a group consisting of tuberculosis, dental caries, periodontitis, and wherein said compound is administered, before, concurrently, or after administration of the drug, and wherein said administration of said compound, and the drug can be in the form of a single dose or multiple doses.

In an embodiment of the present disclosure, there is provided a medicament comprising a drug selected from a group consisting of acarbose, cyclosporine A, its pharmaceutically acceptable salts thereof, and combinations thereof; and at least one compound selected from a group consisting of isoniazid, rifampicin, pyrazinamide, ethambutol, streptomycin, Fe₃O₄ nanocatalyst, clarithromycine, DNase, and pharmaceutically acceptable salts thereof for use in treatment of diseases caused by biofilm forming microorganism: Mycobacterium tuberculosis, and wherein said compound is administered, before, concurrently, or after administration of the drug, and wherein said administration of said compound, and the drug can be in the form of a single dose or multiple doses.

In an embodiment of the present disclosure, there is provided a method of treatment of diseases caused by biofilm forming microorganisms, said method comprising: (a) obtaining acarbose; and (b) administering at least a single dose of a therapeutically relevant amount of acarbose.

In an embodiment of the present disclosure, there is provided a method of treatment of diseases caused by biofilm forming microorganisms, said method comprising: (a) obtaining acarbose; and (b) administering at least a single dose of a therapeutically relevant amount of acarbose, wherein the biofilm forming microorganisms are selected from a group consisting of Mycobacterium smegmatis, Mycobacterium tuberculosis, multidrug resistant M. tuberculosis, extensively drug resistant M. tuberculosis, totally drug resistant variant of M. tuberculosis, and combinations thereof. In another embodiment of the present disclosure, the biofilm forming microorganisms are selected from a group consisting of Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a method of treatment of diseases caused by biofilm forming microorganisms, said method comprising: (a) obtaining a pharmaceutically acceptable salt of acarbose; and (b) administering at least a single dose of a therapeutically relevant amount of a pharmaceutically acceptable salt of acarbose.

In an embodiment of the present disclosure, there is provided a method of treatment of diseases caused by biofilm forming microorganisms, said method comprising: (a) obtaining cyclosporine A; and (b) administering at least a single dose of a therapeutically relevant amount of cyclosporine A.

In an embodiment of the present disclosure, there is provided a method of treatment of diseases caused by biofilm forming microorganisms, said method comprising: (a) obtaining cyclosporine A; and (b) administering at least a single dose of a therapeutically relevant amount of cyclosporine A, wherein the biofilm forming microorganisms are selected from a group consisting of Mycobacterium smegmatis, Mycobacterium tuberculosis, multidrug resistant M. tuberculosis, extensively drug resistant M. tuberculosis, totally drug resistant variant of M. tuberculosis, and combinations thereof. In another embodiment of the present disclosure, the biofilm forming microorganisms are selected from a group consisting of Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a method of treatment of diseases caused by biofilm forming microorganisms, said method comprising: (a) obtaining a pharmaceutically acceptable salt of cyclosporine A; and (b) administering at least a single dose of a therapeutically relevant amount of a pharmaceutically acceptable salt of cyclosporine A.

In an embodiment of the present disclosure, there is provided a method of treatment of diseases caused by biofilm forming microorganisms, said method comprising: (a) obtaining acarbose, cyclosporine A and pharmaceutically acceptable salts thereof; and combinations thereof; (b) obtaining at least one compound (c) administering at least a single dose of a therapeutically relevant amount of acarbose, cyclosporine A and pharmaceutically acceptable salts thereof; and combinations thereof and at least one compound.

embodiment of the present disclosure, there is provided a method of treatment of diseases caused by biofilm forming microorganisms, said method comprising: (a) obtaining acarbose, cyclosporine A and pharmaceutically acceptable salts thereof; and combinations thereof; (b) obtaining at least one compound (c) administering at least a single dose of a therapeutically relevant amount of acarbose, cyclosporine A and pharmaceutically acceptable salts thereof; and combinations thereof and at least one compound, wherein the biofilm forming microorganisms are selected from a group consisting of Mycobacterium smegmatis, Mycobacterium tuberculosis, multidrug resistant M. tuberculosis, extensively drug resistant M. tuberculosis, totally drug resistant variant of M. tuberculosis, and combinations thereof. In another embodiment of the present disclosure, the biofilm forming microorganisms are selected from a group consisting of Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

In an embodiment of the present disclosure, there is provided a method of treatment of diseases caused by biofilm forming microorganisms, said method comprising: (a) obtaining acarbose, cyclosporine A and pharmaceutically acceptable salts thereof; and combinations thereof; (b) obtaining at least one compound; and (c) administering at least a single dose of a therapeutically relevant amount of acarbose, cyclosporine A and pharmaceutically acceptable salts thereof; and combinations thereof and at least one compound, wherein said diseases are selected from a group consisting of tuberculosis, dental caries, and periodontitis.

In an embodiment of the present disclosure, there is provided a method of treatment of diseases caused by biofilm forming microorganisms, said method comprising: (a) obtaining acarbose, cyclosporine A and pharmaceutically acceptable salts thereof; and combinations thereof; (b) obtaining at least one compound; and (c) administering at least a single dose of a therapeutically relevant amount of acarbose, cyclosporine A and pharmaceutically acceptable salts thereof; and combinations thereof and at least one compound, wherein said disease is tuberculosis.

In an embodiment of the present disclosure, there is provided a method of treatment of diseases caused by biofilm forming microorganisms, said method comprising: (a) obtaining acarbose, cyclosporine A and pharmaceutically acceptable salts thereof; and combinations thereof; (b) obtaining at least one compound; and (c) administering at least a single dose of a therapeutically relevant amount of acarbose, cyclosporine A and pharmaceutically acceptable salts thereof; and combinations thereof and at least one compound, wherein said disease is dental caries.

In an embodiment of the present disclosure, there is provided a method of treatment of diseases caused by biofilm forming microorganisms, said method comprising: (a) obtaining acarbose; (b) obtaining at least one compound; and (c) administering at least a single dose of a therapeutically relevant amount of acarbose and at least one compound.

In an embodiment of the present disclosure, there is provided a method of treatment of diseases caused by biofilm forming microorganisms, said method comprising: (a) obtaining a pharmaceutically acceptable salt of acarbose; (b) obtaining at least one compound; and (c) administering at least a single dose of a therapeutically relevant amount of a pharmaceutically acceptable of acarbose and at least one compound.

In an embodiment of the present disclosure, there is provided a method of treatment of diseases caused by biofilm forming microorganisms, said method comprising: (a) obtaining cyclosporine A; (b) obtaining at least one compound; and (c) administering at least a single dose of cyclosporine A and at least one compound.

In an embodiment of the present disclosure, there is provided a method of treatment of diseases caused by biofilm forming microorganisms, said method comprising: (a) obtaining a pharmaceutically acceptable salt of cyclosporine A; (b) obtaining at least one compound; and (c) administering at least a single dose of a therapeutically relevant amount of a pharmaceutically acceptable of cyclosporine A and at least one compound.

In an embodiment of the present disclosure, there is provided a method of treatment of diseases caused by biofilm forming microorganisms, said method comprising: (a) obtaining a drug selected from a group consisting of acarbose, cyclosporine A, pharmaceutically acceptable salts thereof; and combinations thereof; (b) obtaining a compound selected from a group consisting of isoniazid, rifampicin, pyrazinamide, ethambutol, streptomycin, Fe₃O₄ nanocatalyst, clarithromycine, and DNase; and (c) administering at least a single dose of a therapeutically relevant amount of a drug selected from a group consisting of acarbose, cyclosporine A, pharmaceutically acceptable salts thereof; and combinations thereof and at least one or more of said compounds and pharmaceutically acceptable salts; and combinations thereof.

In an embodiment of the present disclosure, there is provided a method of treatment of diseases caused by biofilm forming microorganisms, said method comprising: (a) obtaining a drug selected from a group consisting of acarbose, cyclosporine A, pharmaceutically acceptable salts thereof; and combinations thereof; (b) obtaining a compound selected from a group consisting of isoniazid, rifampicin, pyrazinamide, ethambutol, streptomycin, Fe₃O₄ nanocatalyst, clarithromycine, and DNase; and (c) administering at least a single dose of a therapeutically relevant amount of a drug selected from a group consisting of acarbose, cyclosporine A, pharmaceutically acceptable salts thereof; and combinations thereof and at least one or more of the said compounds and pharmaceutically acceptable salts; and combinations thereof, wherein said diseases are selected from the group consisting of tuberculosis, dental caries, and periodontitis.

In an embodiment of the present disclosure, there is provided a method of treatment of diseases caused by biofilm forming microorganisms, said method comprising: (a) obtaining a drug selected from a group consisting of acarbose, cyclosporine A, pharmaceutically acceptable salts thereof; and combinations thereof; (b) obtaining a compound selected from a group consisting of isoniazid, rifampicin, pyrazinamide, ethambutol, streptomycin, Fe3O4 nanocatalyst, clarithromycine, and DNase; and (c) administering at least a single dose of a therapeutically relevant amount of a drug selected from the group consisting of acarbose, cyclosporine A, pharmaceutically acceptable salts thereof; and combinations thereof and at least one or more of said compounds and pharmaceutically acceptable salts; and combinations thereof, wherein said disease is tuberculosis.

In an embodiment of the present disclosure, there is provided a method of treatment of diseases caused by biofilm forming microorganisms, said method comprising: (a) obtaining a drug selected from a group consisting of acarbose, cyclosporine A, pharmaceutically acceptable salts thereof; and combinations thereof; (b) obtaining a compound selected from a group consisting of isoniazid, rifampicin, pyrazinamide, ethambutol, streptomycin, Fe3O4 nanocatalyst, clarithromycine, and DNase; and (c) administering at least a single dose of a therapeutically relevant amount of a drug selected from a group consisting of acarbose, cyclosporine A, pharmaceutically acceptable salts thereof; and combinations thereof and at least one or more of the said compounds and pharmaceutically acceptable salts; and combinations thereof, wherein said disease is dental caries.

In an embodiment of the present disclosure, there is provided a method of treatment of diseases caused by biofilm forming microorganisms, said method comprising: (a) obtaining acarbose; (b) obtaining isoniazid; and (c) administering at least a single dose of a therapeutically relevant amount of acarbose and isoniazid.

In an embodiment of the present disclosure, there is provided a method of treatment of diseases caused by biofilm forming microorganisms, said method comprising: (a) obtaining a pharmaceutically acceptable salt of acarbose; (b) obtaining isoniazid; and (c) administering at least a single of a therapeutically relevant amount of a pharmaceutically acceptable amount of acarbose and isoniazid.

In an embodiment of the present disclosure, there is provided a method of treatment of diseases caused by biofilm forming microorganisms, said method comprising: (a) obtaining acarbose; (b) obtaining ethambutol; and (c) administering at least a single dose of a therapeutically relevant amount of acarbose and ethambutol.

In an embodiment of the present disclosure, there is provided a method of treatment of diseases caused by biofilm forming microorganisms, said method comprising: (a) obtaining a pharmaceutically acceptable salt of acarbose; (b) obtaining ethambutol; and (c) administering at least a single dose of a therapeutically relevant amount of acarbose and ethambutol.

In an embodiment of the present disclosure, there is provided a method of treatment of diseases caused by biofilm forming microorganisms, said method comprising: (a) obtaining cyclosporine A; (b) obtaining isoniazid; and (c) administering at least a single of a therapeutically relevant amount of cyclosporine A and isoniazid.

In an embodiment of the present disclosure, there is provided a method of treatment of diseases caused by biofilm forming microorganisms, said method comprising: (a) obtaining a pharmaceutically acceptable salt of cyclosporine A; (b) obtaining isoniazid; and (c) administering at least a single of a therapeutically relevant amount of a pharmaceutically acceptable salt of cyclosporine A and isoniazid.

In an embodiment of the present disclosure, there is provided a method of treatment of diseases caused by biofilm forming microorganisms, said method comprising: (a) obtaining cyclosporine A; (b) obtaining ethambutol; and (c) administering at least a single dose of a therapeutically relevant amount of cyclosporine A and ethambutol.

In an embodiment of the present disclosure, there is provided a method of treatment of diseases caused by biofilm forming microorganisms, said method comprising: (a) obtaining a pharmaceutically acceptable salt of cyclosporine A; (b) obtaining ethambutol; and (c) administering at least a single dose of a therapeutically relevant amount of a pharmaceutically acceptable salt of cyclosporine A and ethambutol.

Although the subject matter has been described in considerable detail with reference to certain preferred embodiments thereof, other embodiments are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may vary.

### Experiment 1

### Biofilm Formation Assay

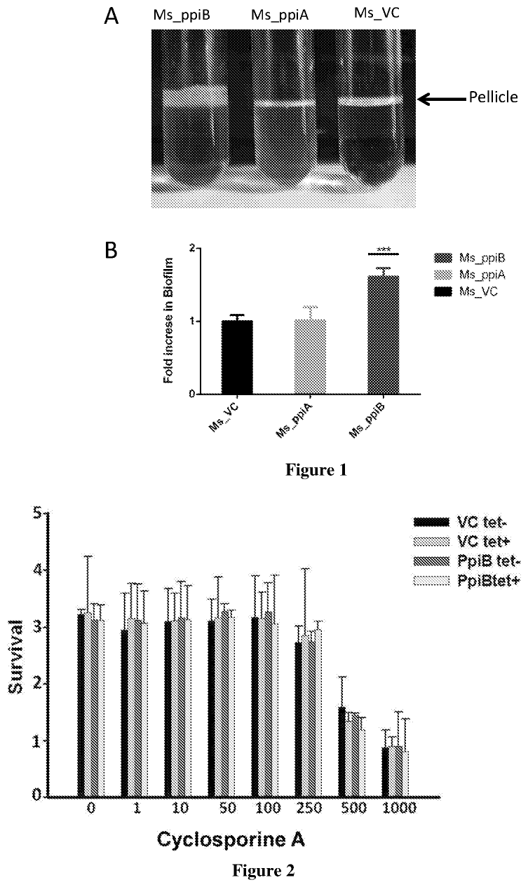

M. tuberculosis PpiB is a glycosylated protein which has a potential lipid modification site (lipobox motif). The biofilm forming potential of overexpressed M. tuberculosis PpiB in M. smegmatis (Ms_PpiB) was compared with M. smegmatis containing empty vector (Ms_VC) (FIG. 1). Biofilm formation in M. smegmatis (Ms_PpiB) was induced by culturing M. smegmatis cells, overexpressing M. tuberculosis PpiB under an anhydrotetracycline inducible promoter. M. smegmatis (Ms_VC) cultured in absence (VC Tet−) or presence (VC Tet+) of anhydrotetracycline or M. smegmatis cultured in absence (Ms_PpiB Tet−) of anhydrotetracycline were used as control and compared against biofilm formed in M. smegmatis cultured in presence of anhydrotetracycline (Ms_PpiB Tet+). Briefly, M. smegmatis was cultured in Middlebrook 7H9 agar broth supplemented with 10% Middlebrook OADC, henceforth referred as 7H9 media. Protein expression was induced by addition of 20 ng/ml of anhydrotetracycline and cells were incubated for 48 hours. The culture was then diluted to an OD of 0.08 in 7H9 media and pipetted in each well of a 96-well microtitre plate. For further studies in this disclosure, various concentrations of different biofilm inhibitors (cyclosporine A and acarbose) were added into each well of a 96-well microtitre plate, in addition to anhydrotetracycline. The plates were then covered and incubated at 37° C. for 7 days. After incubation, the content of the wells was aspirated out and stained by adding 125 μl of 0.1% Crystal Violet (w/v) solution to each well. Each well was then washed thrice with water followed by addition of 30% acetic acid. The plates were subsequently incubated for 10 to 15 min. at room temperature and absorbance was taken at 550 nm.

The biofilm assay was extrapolated to assess the effect of cyclosporine A on M. tuberculosis H37Rv strain. Briefly, H37Rv cells were inoculated at an OD=0.6 through the walls of the test tube containing 7H9 media. The test tube containing control and Cyclosporin A (100 μg/ml) were incubated undisturbed in static phase for 4 weeks. The pellicle formed at the liquid air interface was evaluated for biofilm formation.

As is illustrated in FIG. 1, PpiB overexpressing M. smegmatis (Ms_PpiB) exhibited enhanced biofilm and pellicle formation, as compared to PpiA over expressing M. smegmatis (Ms_PpiA) and M. smegmatis containing empty vector (Ms_VC). This indicates that M. tuberculosis PpiB is a protein involved in biofilm formation.

As is illustrated from FIG. 14, Mycobacterium H37Rv treated with cyclosporine A (100 μg/ml) exhibited reduced pellicle formation, as compared to control cells. This indicates that Cyclosporin A suppresses biofilm formation in H37Rv cells.

### Experiment 2

Effect of Cyclosporine A and Acarbose on the Viability of M. smegmatis Over-Expressing M. tuberculosis PpiB Protein

The effect of cyclosporine A and acarbose on the viability of M. smegmatis over-expressing M. tuberculosis PpiB under tetracycline inducible promoter was scored using colorimetric alamar blue staining as described elsewhere (Taneja et al. J Antimicrob Chemother 2007; 60(2): 288-293). M. smegmatis cells, in log phase were induced with anhydrotetracycline, cultured for 24 hours and cells from secondary culture of bacteria were seeded in a 96 well plate at an initial OD of 0.08 in 7H9 media. Cells were incubated with/without anhydrotetracycline in presence of various concentration of cyclosporine A or acarbose. After 68 hours, alamar blue reagent was added to the cells and incubated for an additional 3-4 hours. Viability was measured as the variance between absorbances at 570 nm and 600 nm Blanks for spectrophotometry were provided by wells containing medium alone and alamar blue. All assays were performed in triplicate.

At 500 μg/ml and above, cyclosporine A significantly inhibited the viability of M smegmatis, and this effect was much enhanced in PpiB cultures treated with anhydrotetracycline (Tet+) as compared to the controls without anhydrotetracycline (Tet⁻) (FIG. 2).

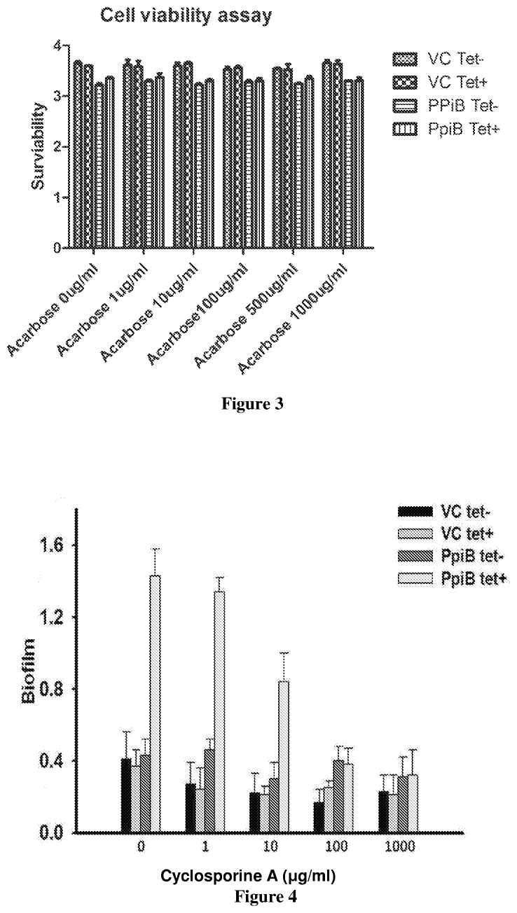

Further, FIG. 3 shows that up to 1 mg/ml concentration of acarbose did not have any effect on the viability of M. smegmatis cells either expressing M. tuberculosis PpiB (PpiB Tet+) or vector control (VC Tet+).

### Experiment 3

Effect of Cyclosporine A and Acarbose on PpiB Mediated Biofilm Formation in M. smegmatis Over-Expressing M. tuberculosis PpiB Protein

In order to examine the inhibitory role of cyclosporine A or acarbose on biofilm formation, M. smegmatis was cultured in presence of various concentrations of cyclosporine A or acarbose as described above. After 7 days, cells were removed and the biofilm formed at the solid liquid interface was stained using 0.1% solution of crystal violet for 10-15 minutes, as described elsewhere (O'Toole J Vis Exp 2011; 47:2437). Crystal violet was dissolved in 30% acetic acid and quantified colorimetrically at 550 nm as an estimate of the biofilm formed.

As illustrated in FIG. 4, anhydrotetracycline induced PpiB led to greater formation of biofilm in PpiB expressing M. smegmatis (Ms_PpiB Tet+) as compared to PpiB uninduced (Ms_PpiB Tet−) or in Vector control (Ms_VC Tet−/Tet+). Treatment with cyclosporine A above 100 μg/ml significantly reduced biofilm formation in Ms_PpiB (Tet+). cyclosporine A could therefore bind to PpiB and inhibit the activity of PpiB which in turn reduces biofilm formation.

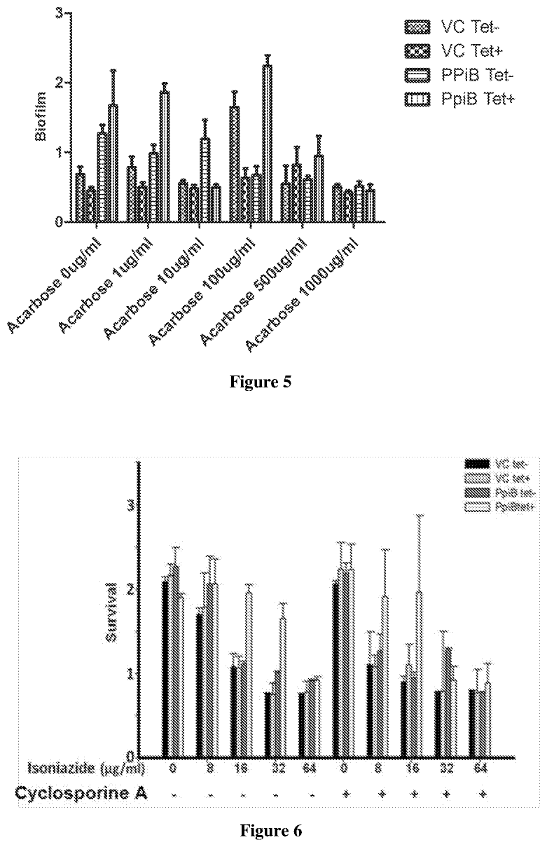

Additionally, as depicted in FIG. 5, there is a significant inhibition in the biofilm formation above 500 μg/ml of acarbose treatment in anhydrotetracycline induced-PpiB overexpressing M. smegmatis cells (Ms_PpiB Tet+) as compared to M. smegmatis containing empty vector (Ms_VC). Each experiment was performed three times independently and statistical analysis was done using one-way Anova.

Thus, both acarbose and cyclosporine A can inhibit biofilm formation in M. smegmatis overexpressing M. tuberculosis PpiB protein.

### Experiment 4

Effect of Isoniazide and Ethambutol on Survival of M. smegmatis in the Presence and Absence of Cyclosporine A or Acarbose

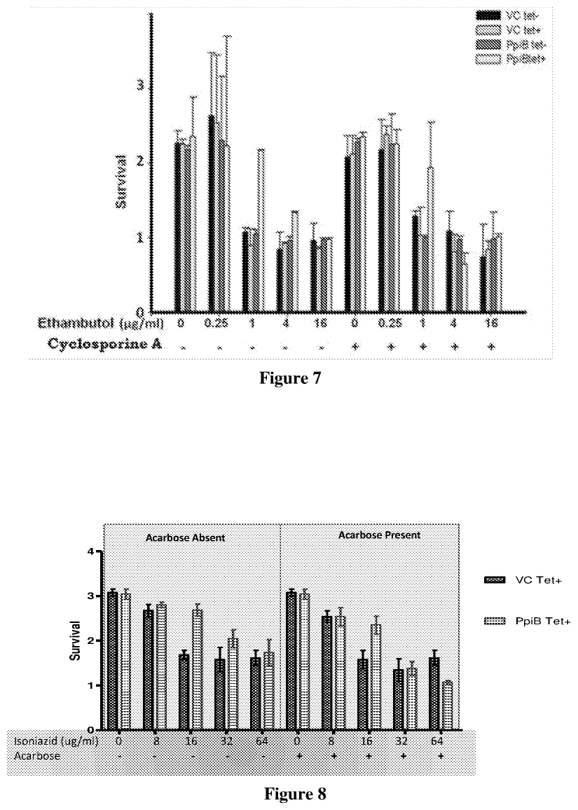

Biofilm formation contributes to increased drug tolerance. In order to examine the effect of cyclosporine A or acarbose on biofilm mediated drug tolerance in M. smegmatis, cells were incubated either in the absence or presence of cyclosporine A or acarbose and biofilm formation assay was performed as described previously. At the end of 7 days of culture, cells were treated with isoniazide (0, 8, 16, 32, 64 μg/ml) or ethambutol (0, 0.25, 1, 4. 16 μg/ml) for additional 72 hrs. The viability of cells was assessed using alamar blue reagent. Results in FIG. 6 indicate that PpiB-induced biofilm increases the tolerance of M. smegmatis towards isoniazide. The minimum inhibitory concentration (MIC) of isoniazide in absence of cyclosporine A was 16 μg/ml for VC(tet−/tet+) and PpiB (tet−) cultures whereas it was 64 μg/ml in PpiB (tet+) suggesting that PpiB induced biofilm increases the tolerance of M. smegmatis towards isoniazide. The MIC of isoniazide in the presence of cyclosporine A treatment was 8 μg/ml for VC(tet−/tet+) and PpiB (tet−) cultures whereas it was 32 μg/ml in PpiB (tet+) suggesting the role of cyclosporine A on reducing biofilm formation which in turn increases the susceptibility of M. smegmatis towards isoniazide. Similar results were observed with ethambutol in presence of cyclosporine A. As seen in FIG. 7, ethambutol inhibits PpiB (Tet+) culture in the absence of cyclosporine A treatment at 4 μg/ml and this effect was enhanced in the presence of cyclosporine A.

Similar to the results obtained above, the MIC of isoniazide in the absence of acarbose was 16 μg/ml for VC (Tet+) and 64 μg/ml for PpiB (Tet+) cells. As depicted in FIG. 8, in the presence of acarbose, MIC of isoniazide decreased to 32 μg/ml for PpiB (Tet+), thus proving that the effect of acarbose on biofilm reduction consequently reduces the tolerance of M. smegmatis towards isoniazide.

These results propose both cyclosporine A and acarbose as leading compounds to support anti-TB drugs to reduce the MIC in MDR tuberculosis in combination with either isoniazid or ethambutol.

### Experiment 5

In Silico Interaction Study of PpiB with US FDA Approved Drugs

Crystallographic structure of PpiB is not present at Protein Data Bank so it was modelled through Phyre2 server (Kelley et al, Nat Protocols, 2015; 10, 845-858). Protein sequence of PpiB of M. tuberculosis (strain ATCC 25618/H37Rv) was obtained from UniprotKB database [P9WHW1] (Consortium U, Nucleic Acids Research, 2014; D204-12). Molecular dynamics (MD) was performed for the modelled structure through GROMACS v5.0 (Abraham et al, GROMACS User manual, 2014) using OPLS2005 force field. Firstly, the modelled protein structure was solvated into a triclinic box using spc water model. Then, the system was neutralized, energy minimized as well as equilibrated through NVT and NPT ensemble then well equilibrated system was processed to production run of MD for 40 nanoseconds (ns). Protein structure model validation was done through protein structure validation software suite (PSVS) (Bhattacharya et al, Proteins: Structure, Function, and Bioinformatics, 2007; 66: 778-95) that includes the large set of quality and constraint analysis. It includes the stereochemical quality analysis and model compatibility with own amino acid sequence through Verify_3 D (Luthy et al, Nature, 1992; 356(6364):83-5; Eisenberg et al, Methods Enzymol., 1997; 277:396-404). and all-atom contact and covalent geometry analysis through MolProbity (Chen et al, Acta crystallographica D, 2010; 66(Pt 1): 12-21). Representative frame was selected from the simulation trajectory for docking analysis.

Docking study of the PpiB protein with acarbose and other FDA approved drugs was performed through Glide module of Schrodinger (Schrödinger et al, LLC, New York, USA, 2009). For this the protein structure was prepared through Protein preparation wizard. Protein was pre-processed by assigning bond orders, adding hydrogen, deleting water molecules further than 5 Å distance as well as converting all selenomethionines to methionines. Uncapped N and C termini were capped by ACE (N-acetyl) and NMA (N-methyl amide) respectively. Hydrogen bond assignment was done using ProtAssign that optomizes hydroxyl, Asparagine, Glutamine and Histidine states. Impref minimization was done using 0.30 Å RMSD and OPLS 2005 force field.

For PpiB ligand binding studies and identification of possible inhibitors of PpiB, the approved drugs were prepared through LigPrep module of Schrodinger suite using OPLS2005 force field. All possible states at pH 7+/−2 were generated using ionizer and retaining specific chiralities of the molecules. At most, 32 ligands were generated per ligand molecule and the output was saved in maestro format for the docking purpose.

The active site residues were selected on the basis of prediction through 3D Ligand site server that performs the model based as well as the sequence based prediction. Both strategies were used to get the active site residues i.e. Pro135, Asn223 and Pro225. Confirmation of docking accuracy was also carried out using another platform AutoDock Tools (Morris et al, J Comput. Chem., 2009; 30(16): 2785-2791) and inhibition constants were also estimated.

PpiB amino acid sequence was submitted to Phyre2 and intensive mode was chosen for model building as it uses multiple templates and ab-initio techniques. The modeled structure was found having an overall 98% residue in the allowed regions. Global quality Z-score values for MolProbityClash score is recommended to be greater than −3 for high quality structures, and our model scored −1.23, suggesting an adequate model. Verify 3D also corroborated the reasonable quality of the model. Thus, the PpiB model satisfied the structure quality parameters of an acceptable protein structure model.

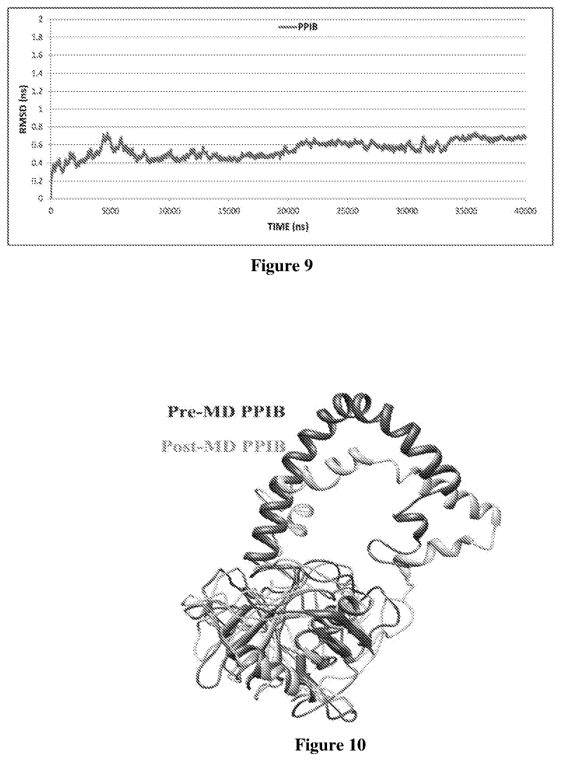

The structure obtained from the server was processed for molecular dynamics simulation to get a stable starting structure for molecular docking studies. RMSD analysis for the PpiB protein showed a stable trajectory from 20 ns to 40 ns with small deviations in the range of 0.2 nm (FIG. 9). The starting structure from the 40000 ps trajectory was obtained through cluster analysis using GROMACS by gromos method (Daura et al, Proteins: structure, function, and bioinformatics, 1999; 34: 269-280). FIG. 10 shows a superimposition of Pre-MD and Post-MD PpiB protein models.

Docking was performed in two steps i.e. HTVS (high throughput virtual screening) (Sastry et al, J Comput Aided Mol Des, 2013; 27: 221) and XP (extra precision) docking (Friesner et al, J. Med. Chem., 2006; 49(21), 6177-6196). HTVS docking method filtered out the compounds with low binding energy (Singh et al, Structural Chemistry, 2015; 27: 993-1003). HTVS docking method filtered out the compounds with low binding energy. The compounds having a docking score greater than −5 in HTVS were used for XP docking protocol. An XP score greater than −8 showing strong binding was displayed by many of the USFDA approved drugs and this is given in Table 1.

TABLE 1

| | DRUG | GENERIC | | DOCKING | |
|---|---|---|---|---|---|
| | BANK ID | NAME | PRODUCTS | SCORE | FUNCTIONS |
| 1. | DB00284 | acarbose | acarbose; Glucobay; | −13.2704 | Reversible binding to |
| | | | Precose | | pancreatic |
| | | | | | alpha-amylase & |
| | | | | | membrane-bound intestinal |
| | | | | | alpha-glucoside hydrolases. |
| | | | | | Used for treatment of |
| | | | | | diabetes type II. |
| 2. | DB08995 | Diosmin | | −12.1824 | It is a semisynthetic |
| | | | | | drug used for the |
| | | | | | treatment of venous |
| | | | | | diseases. |
| 3. | DB01092 | Ouabain | | −9.75823 | It inhibits |
| | | | | | Na—K- ATPase |
| | | | | | membrane pump and used in |
| | | | | | treatment of atrial |
| | | | | | fibrillation & heart |
| | | | | | failure. |
| 4. | DB08816 | Ticagrelor | Brilinta | −9.66564 | It blocks ADP |
| | | | | | receptors. |
| | | | | | Used for prevention of |
| | | | | | thrombotic events such |
| | | | | | as stroke & heart |
| | | | | | failure. |
| 5. | DB03147 | Flavin adenine | | −9.45861 | It is used to treat eye |
| | | dinucleotide | | | diseases due to vitamin |
| | | | | | B2 deficiency. |
| 6. | DB00287 | Travoprost | Izba; Travatan Z | −9.38875 | It is a selective |
| | | | | | prostanoid receptor |
| | | | | | agonist that is used to |
| | | | | | reduce elevated |
| | | | | | intraocular pressure. |
| 7. | DB00987 | Cytarabine | Cytarabine; Cytosar | −8.99202 | It acts by direct DNA |
| | | | | | damage as well as |
| | | | | | incorporation into |
| | | | | | DNA. Used in |
| | | | | | treatment of different |
| | | | | | forms of leukaemia. |
| 8. | DB00256 | Lymecycline | | −8.72875 | Inhibits cell growth via |
| | | | | | inhibition of translation |
| | | | | | and used for treatment |
| | | | | | of infections & acne. |
| 9. | DB00928 | Azacitidine | Azacitidine; Vidaza | −8.6861 | Inhibits the DNA |
| | | | | | methyltransferase at |
| | | | | | low doses while |
| | | | | | incorporates into DNA |
| | | | | | and RNA at high doses, |
| | | | | | resulting in cell death. |
| | | | | | Used for treatment of |
| | | | | | patients with |
| | | | | | French-American-British |
| | | | | | myelodysplastic |
| | | | | | syndrome subtypes. |
| 10. | DB01421 | Paromomycin | | −8.52744 | Inhibits protein |
| | | | | | synthesis via 16S |
| | | | | | ribosomal RNA |
| | | | | | binding. |
| | | | | | Used for treatment of |
| | | | | | acute as well as chronic |
| | | | | | intestinalamebiasis. |
| 11. | DB00131 | Adenosine | | −8.34908 | Dietary supplement to |
| | | monophosphate | | | boost immune activity. |
| | | (AMP) | | | Also used as substitute |
| | | | | | sweetener for |
| | | | | | low-calorie diet. |
| 12. | DB00694 | Daunorubicin | Daunorubicin | −8.34677 | It forms complexes |
| | | | Hydrochloride; | | with DNA thereby |
| | | | Daunoxome | | having cytotoxic and |
| | | | | | antimitotic |
| | | | | | activity. Used in the |
| | | | | | treatment of |
| | | | | | nonlymphocyticleukaemia. |
| 13. | DB00445 | Epirubicin | Ellence | −8.32349 | Inhibits nucleic acid |
| | | | | | and protein synthesis |
| | | | | | via different |
| | | | | | mechanisms. |
| | | | | | Used in adjuvant |
| | | | | | therapy for patients |
| | | | | | with axillary node |
| | | | | | tumor involvement. |
| 14. | DB00997 | Doxorubicin | Caelyx; Doxil; | −8.32349 | Intercalates between |
| | | | Myocet | | base pairs and inhibits |
| | | | | | topoisomerase II |
| | | | | | activity thereby having |
| | | | | | antimitotic and |
| | | | | | cytotoxic activities. |
| | | | | | Used in treatment of |
| | | | | | acute myeloblastic |
| | | | | | leukemia and acute |
| | | | | | lymphoblastic |
| | | | | | leukemia, |
| 15. | DB00385 | Valrubicin | Valstar; Valtaxin | −8.17939 | An anthracycline that |
| | | | | | affects nucleic acid |
| | | | | | metabolism and used in |
| | | | | | bladder cancer |
| | | | | | treatment. |
| 16. | DB00552 | Pentostatin | Nipent | −8.14385 | A transition state |
| | | | | | inhibitor of |
| | | | | | ADA(adenosine |
| | | | | | deaminase) and used in |
| | | | | | treatment of hairy cell |
| | | | | | leukaemia refractory to |
| | | | | | alpha interferon. |
| 17. | DB08934 | Sofosbuvir | Sovaldi | −8.12207 | A nucleotide analog |
| | | | | | inhibitor, that inhibits |
| | | | | | HCV NS5B |
| | | | | | polymerase and used in |
| | | | | | combination therapy |
| | | | | | or treatment of chronic |
| | | | | | hepatitis C virus. |
| 18. | DB01016 | Glyburide | Diabeta; Euglucon; | −8.10286 | It binds to |
| | | | Glyburide; Glynase | | ATP-sensitive potassium |
| | | | | | channels on pancreatic |
| | | | | | cell surface and used as |
| | | | | | an adjunct to diet for |
| | | | | | lowering blood glucose |
| | | | | | in patients with |
| | | | | | NIDDM. |
| 19. | DB01073 | Fludarabine | Fludara; Fludarabine; | −8.07998 | It gets converted to |
| | | | Fludarabine Phosphate; | | 2-fluoro-ara-ATP and |
| | | | Oforta | | this metabolite inhibits |
| | | | | | DNA synthesis. |
| | | | | | Used for treatment of |
| | | | | | B-cell chronic |
| | | | | | lymphocytic leukaemia |
| 20. | DB01132 | Pioglitazone | Actos; | −8.0654 | Agonist of peroxisome |
| | | | Pioglitazone; | | proliferator activated |
| | | | Hydrochloride | | receptors (PPAR) and |
| | | | | | used in treatment of |
| | | | | | Type II diabetes |
| | | | | | mellitus. |
| 21. | DB01177 | Idarubicin | Idarubicin; | −8.05196 | Intercalates between |
| | | | Idarubicin | | base pairs and inhibits |
| | | | Hydrochloride | | topoisomerase II |
| | | | | | activity thereby having |
| | | | | | antimitotic and |
| | | | | | cytotoxic activities. |
| | | | | | Used for treatment of |
| | | | | | acute myeloid leukemia |
| | | | | | (AML |
| 22. | DB08907 | Canagliflozin | Invokana | −8.04845 | Inhibitor of (SGLT2) |
| | | | | | Sodium-glucose |
| | | | | | co-transporter 2 and an |
| | | | | | adjunct to diet for |
| | | | | | improving glycemic |
| | | | | | control in adult patients |
| | | | | | of type 2 diabetes |
| | | | | | mellitus. |

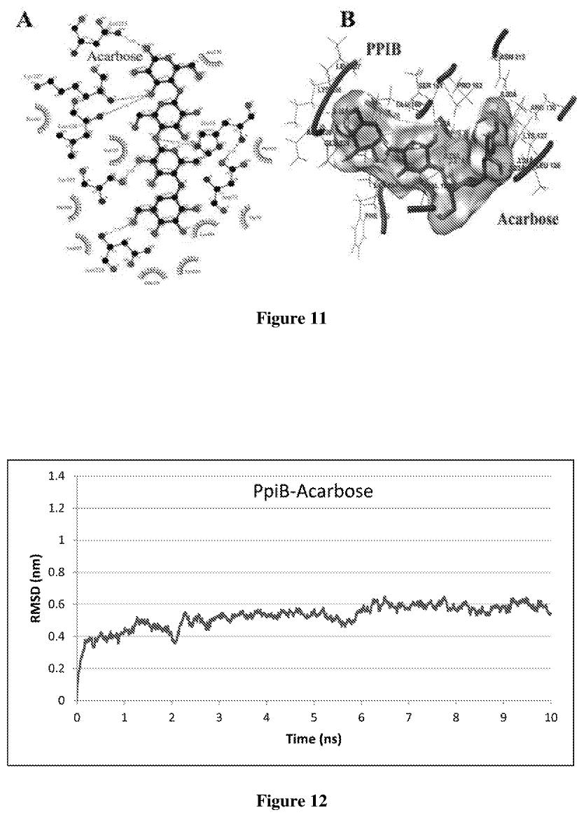

Molecular docking carried out using alternate platform also confirmed high binding energies of acarbose with PpiB. Estimated inhibition constant (Ki) was found in the range of 16-20 μM for acarbose. The interaction plot of the drug with PpiB protein was made using LigPlot⁺ software and represented in FIG. 11 (Laskowski et al, J. Chem. Inf. Model., 2011; 51(10):2778-86). These results provide interesting leads with respect to USFDA approved drugs for their likely value in acting as inhibitors of PpiB, which is associated with enhancement in biofilm formation and by corollary in rendering the pathogen more responsive to drugs.

Molecular dynamics simulations of the PpiB docked complexes with acarbose were also performed using GROMACS v5.0 assigning GROMOS96 (van Gunsteren et al, The {GROMOS96} manual and user guide 1996) 43a1 force field. GOMACS topology for the ligand was generated using the PRODRG (Schuettelkopf et al, Acta Crystal Clographica, 2004; D60, 1355-1363). Docked complexes of PpiB with both the drugs were solvated in a triclinic water boxes using spc water molecules. genion tool was used to make the systems electrically neutral. Systems were subjected to energy minimization by steepest descent method for 50000 ns steps. MD simulation for the complexes was performed in two steps i.e. first the systems were equilibrated and then the production run was carried out. Initially NVT (isothermal-isochoric) and then NPT (isothermal-isobaric) equilibration was done in continuation to stabilize the temperature and pressure of the systems, respectively. Both the equilibration steps were performed for 50 ps time. After attaining the desired temperature and pressure, systems were proceeded for MD production run of 10000 ps/10 ns to attain stable trajectories of the complexes.

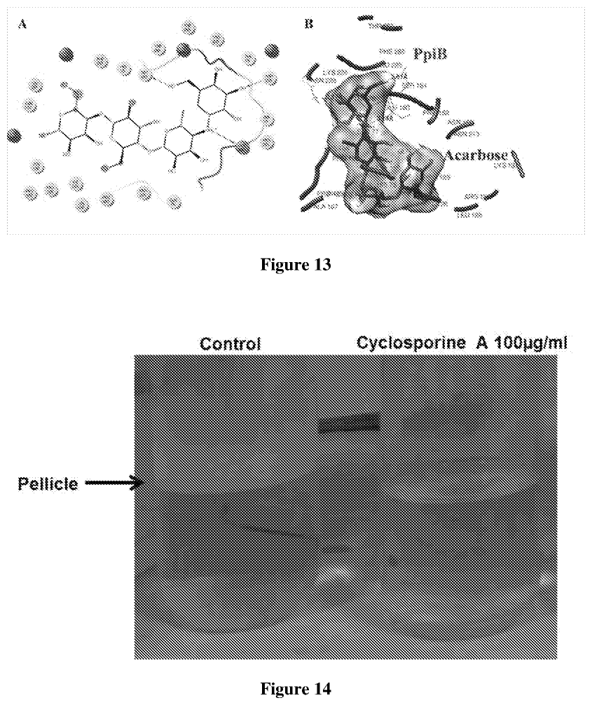

After completion of the 10 ns molecular dynamics the trajectories were analysed. First the trajectories were made compact, protein and ligand complexes were centered to the solvation box and fit analysis was also done with reference to the starting structure. MD trajectory after the fit analysis was used for further analysis of the docked complex structures. RMSD analysis for the complex trajectories was done using GROMACS. It shows the stable trajectory for PpiB-Acabose complex from 6-10 ns with minimal fluctuations in acceptable range as shown in FIG. 12. Representative structures from the stable trajectories were extracted using the cluster analysis approach and the hydrogen and hydrophobic interactions were plotted. Both the complexes show new interactions after MD while retaining few pre-MD interactions represented in FIG. 13.

### Experiment 6

### In Silico Molecular Docking Analysis of M. Tb PpiB

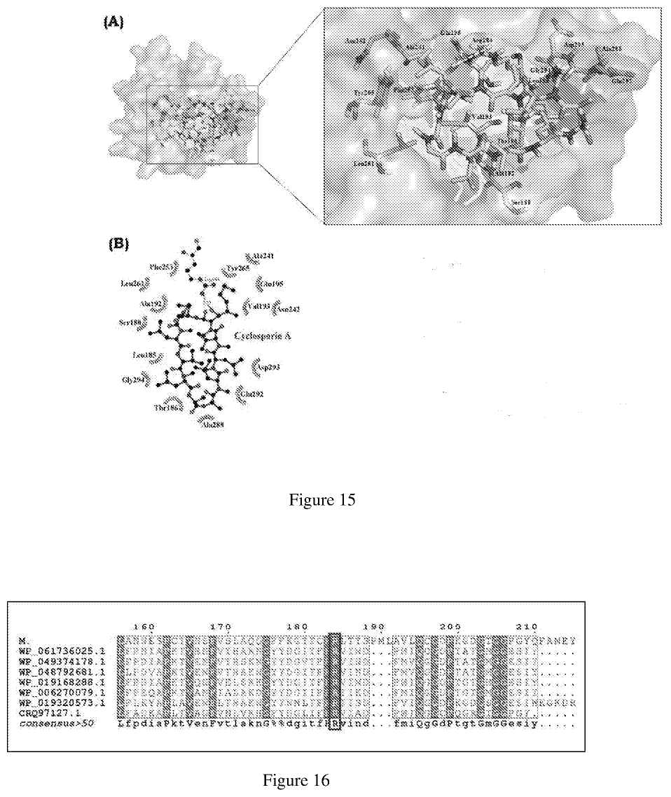

The In silico molecular docking analysis of M. tb PpiB in complex with cyclosporine A was carried out to study the interactions and affinity of compound cyclosporine A with the protein PpiB. The protein structure model of M. tb PpiB was generated by homology modelling technique using the MODELLER version 9.11 (Fiser and Sali, Methods Enzymol 2003: 374: 461-91). The 3D structure of cyclosporine A was obtained from chemical structure database ChemSpider (Pence et al, J. Chem. Educ. 2010; 87(11):1123-1124). Polar hydrogens were added, non-polar hydrogens were merged and computation of Gastegier partial charges was carried out (Gasteiger and Marsilli, 1980; doi:10.1016/0040-4020(80)80168-2) for the protein structure using Autodock Tools 1.5.6 (ADT) package (Morris et al., J Comput Chem 2009; 30(16):2785-2791 before embarking docking analysis. The program AutoDockVina 1.1.2 (Trott and Olson, J Comput Chem 2010; 31(2):455-461) was used for further docking of cyclosporine A at the catalytic active site of PpiB protein. The location of the catalytic site was mapped and deduced from the structure based alignment of related proteins reported earlier (Henriksson et al., Eur J Biochem 2004; 271(20):4107-13). The Arg184 conserved residue from the catalytic centre was used for docking analysis and therefore it was assumed that this protein-drug complex may represent a real entity. Docked complex of protein-ligand was then energy minimized using the GROMACS, version 4.6.5 (Abraham et al., 2015; doi/10.1016/j.softx.2015.06.001 under OPLS-AA force field for optimising the correct conformation of binding mode and stability of the protein-ligand complex. Ligplot (Laskowski and Swindells, J Chem Inf Model 2011) was used for the visualization of the interactions between protein-ligand complex in two-dimensional schematic representations and the program PyMol (www.pymol.org) was used for the preparing cartoon representations of the structures. Results in FIGS. 15A and 15B show multiple interactions between cyclosporine A and M. tb PpiB.

Molecular docking studies of cyclosporine A binding sites in all PpiB homologues expressed in different biofilm forming bacteria were compared. Results in FIG. 16 show that M. tb PpiB (Rv2582) exhibits homology with proteins from other biofilm forming bacteria and possess similar amino acids at binding site of cyclosporine A.

The results demonstrate that Arg184 residue from PpiB, is conserved across the homologous proteins, in some of the cases it is present at different positions. The position of conserved Arginine residue present in different groups of biofilm forming microorganisms, listed in Table 2, in terms of gene bank accession numbers: WP_061736025.1, WP_049374178.1, WP_019168288.1, WP_019320573.1, WP_048792681.1, WP_006270079.1, CRQ97127.1 are Arg59, Arg59, Arg59, Arg107, Arg59, Arg327 and Arg46, respectively. Molecular docking studies of acarbose binding sites in all PpiB homologues expressed in different biofilm forming bacteria were compared. Results in Table 2 show that acarbose could bind to all homologous PpiB proteins expressed in different biofilm forming bacteria, listed in Table 2. Pro63 in M. tb is conserved for acarbose binding in WP_061736025.1, WP_019168288.1, and WP_019320573.1. In WP_048792681.1, WP_006270079.1 and CRQ97127.1 exhibited a nearby Pro33 residue to compensate the binding affinity (Table 2). Presence of conserved amino acids at the cyclosporine A or acarbose binding site in PpiB homologues of several biofilm forming bacteria indicate that the active site of PpiB have largely remained unaltered and hence could prove to be an excellent putative target across bacterial species.

TABLE 2

| | | | | | | Binding | Binding |
|---|---|---|---|---|---|---|---|
| | | | | | | Pocket | Pocket |
| Name of the | Role in | GenBank accession | Homology | | Query | Residues of | Residues of |
| organism | biofilm | number | with PpiB | E-value | coverage | cyclosporine A | acarbose |
| Staphylococcus | Cystic | WP_061736025.1 | 35% | 9e−20 | 54% | His186, | Ala2, |
| aureus | Fibrosis, | | | | | His58, | Asn3, |
| | Pacemakers, | | | | | Pro184, | Tyr4, |
| | Prosthetic | | | | | Asp187, | Pro5, |
| | heart valves, | | | | | Lys183, | Gln6, |
| | Contact | | | | | Val160, | Leu7, |
| | Lenses, | | | | | Arg59, | Gly14, |
| | Orthopaedic | | | | | Leu185, | Glu15, |
| | implants | | | | | Val178, | Ile16, |
| | | | | | | Lys177 | Pro33, |
| | | | | | | | Asn34, |
| | | | | | | | Pro37, |
| | | | | | | | Lys38, |
| | | | | | | | Glu41, |
| | | | | | | | Tyr82 |
| Staphylococcus | Prosthetic | WP_049374178.1 | 33% | 8e−18 | 53% | Tyr186, | Lys9 |
| epidermidis | heart valves, | | | | | Asp187, | Asn14 |
| | Wounds, | | | | | Val160, | Ile16 |
| | | | | | | Pro184, | Lys17 |
| | | | | | | His58, | Lys30 |
| | | | | | | Val185, | Phe32 |
| | | | | | | Arg59, | Pro33 |
| | | | | | | Val178, | Asp34 |
| | | | | | | Ile61, | Glu197 |
| | | | | | | Lys177 | |
| Staphylococcus | Wounds | WP_019168288.1 | 31% | 3e−19 | 54% | Asp187, | Met1, |
| intermedius | | | | | | Tyr186, | Thr2, |
| | | | | | | Pro184, | Tyr4, |
| | | | | | | His58, | Pro5, |
| | | | | | | Met185, | Gln6, |
| | | | | | | Arg59, | Leu7, |
| | | | | | | Val178, | Lys9, |
| | | | | | | Val60, | Gln12, |
| | | | | | | Lys177 | Glu13, |
| | | | | | | | Pro37, |
| | | | | | | | Lys39, |
| | | | | | | | Gln41, |
| | | | | | | | Tyr82, |
| | | | | | | | Glu87 |
| Streptococcus | Dental | WP_019320573.1 | 33% | 1e−22 | 56% | Gln238, | Leu5 |
| mutans | biofilm, | | | | | Lys240, | Val8 |
| | Orthopaedic | | | | | Lys236, | Leu9 |
| | implants, | | | | | Thr104, | Phe12 |
| | Wounds, | | | | | Ser234, | Lys41 |
| | Prosthetic | | | | | His106, | Lys43 |
| | heart valves, | | | | | Gly235, | Leu44 |
| | Pacemakers | | | | | Gln233, | Lys45 |
| | | | | | | Asn232, | Gln46 |
| | | | | | | Pro119, | Leu47 |
| | | | | | | Lys120, | Glu63 |
| | | | | | | Gly121, | Ala64 |
| | | | | | | Gln171, | Leu81 |
| | | | | | | Arg107 | Lys82 |
| | | | | | | | Pro85 |
| | | | | | | | Val88 |
| | | | | | | | Glu89 |
| | | | | | | | Leu92 |
| | | | | | | | Asp251 |
| Staphylococcus | Contact | WP_048792681.1 | 31% | 2e−18 | 53% | His58, | Asn3 |
| saprophyticus | lenses | | | | | His186, | Tyr4 |
| | | | | | | Asp187, | Pro5 |
| | | | | | | Pro184, | Gln6 |
| | | | | | | Leu185, | Leu7 |
| | | | | | | Arg59, | Ile16 |
| | | | | | | Val60, | Lys17 |
| | | | | | | Val178, | Lys30 |
| | | | | | | Lys177 | Leu31 |
| | | | | | | | Leu32 |
| | | | | | | | Pro33 |
| | | | | | | | Asp34 |
| | | | | | | | Val35 |
| | | | | | | | Glu93 |
| | | | | | | | Gln162 |
| | | | | | | | Asp197 |
| Streptococccus | Dental | WP_006270079.1 | 32% | 1e−19 | 52% | Ala374, | Leu 140, |
| constellatus | biofilm | | | | | His424, | Pro 141, |
| | | | | | | Asn373, | Val 178, |
| | | | | | | Gly343, | Arg 179, |
| | | | | | | Met344, | Trp 180, |
| | | | | | | His326, | Glu 218, |
| | | | | | | Met450, | Leu 219, |
| | | | | | | Asp451, | Gly 235, |
| | | | | | | Lys452, | Ile 236, |
| | | | | | | Thr342, | Ser 237, |
| | | | | | | Arg327 | His 238, |
| | | | | | | | Lys 239, |
| | | | | | | | Lys 242 |
| Pseudomonas | Cystic | CRQ97127.1 | 38% | 1e−21 | 45% | Arg46, | Lys 22, |
| aeruginosa | Fibrosis, | | | | | Gly141, | Ala 23, |
| | Wounds, | | | | | Leu139, | Pro 24, |
| | Contact | | | | | Asp142, | Leu 25, |
| | Lenses, | | | | | His45, | Glu 71, |
| | Orthopaedic | | | | | Phe44, | Asp 72, |
| | implants, | | | | | Glu140, | Glu 73, |
| | Breast | | | | | Val47, | Lys 74, |
| | implants, | | | | | Gly131, | Phe 115 |
| | | | | | | Ile48, | |
| | | | | | | Asp132 | |

### Experiment 7

### Amino Acid Sequence Analysis

The amino acids sequences of M. tb PpiA (GenBank Accession number: CCP42731.1), M. tb PpiB (GenBank Accession number: CCE38048.1), E. coli peptidyl proliyl isomerase (GenBank Accession number: EDV66960.1) and peptidyl-prolyl isomerase RopA (trigger factor) of Streptococcus mutans (GenBank Accession number: AAN57875.1) were downloaded from the National Center for Biotechnology Information (https://www.ncbi.nlm.nih.gov). To examine the difference in sequences of M. tb PpiB with other peptidyl prolylisomerses M. tb PpiA and E. coli peptidyl proliyl isomerase, multiple sequence alignment of these sequences was done using MULTIALIN algorithm (Corpet, Mucleic Acid Res. 1988; 16(22); 10881-10890). To examine the similarity of M. tb PpiB with the peptidyl-prolylisomerase (trigger factor) involved in biofilm formation, multiple sequence alignment of amino acids sequence of M. tb PpiA, M. tb PpiB and RopA was carried out. The sequence homology search of M. tb PpiB was done using BLASTp in known biofilm forming bacteria such as Staphylococcus aureus, Staphylococcus epidermidis, Staphylococcus intermedius, Streptococcus mutans, Staphylococcus saprophyticus, Streptococcus constellatus on NCBI website. M. tb PpiB amino acids sequence was used as queries in BLASTp analyses against the NCBI non-redundant protein database of the specific bacteria to find their similar homologues.

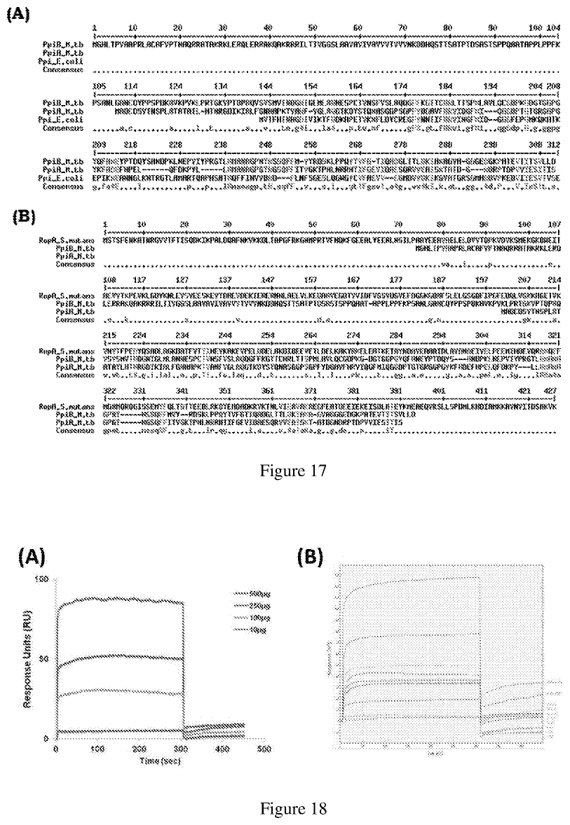

Results in FIG. 17A show that M. tb PpiA or PpiB exhibit high degree of similarity in conserved amino acids found in E. coli Ppiase. M. tb PpiB also possesses an extended sequence of 100 amino acids in the N terminal end and is absent in either M. tb PpiA or E. coli Ppiase. Results in FIG. 17B show that, M. tb Ppiase exhibit high degree of sequence similarity with trigger factor proteins (RopA) expressed in S. mutans. Previous studies provided evidence that M. tb PpiB possess chaperonic acivity and plays crucial role in protein stability and protein folding (Pandey et al., PloS One 2016; 11(3):e150288). The importance of chaperons in aiding biofilm formation in S. mutans is already known. By virtue of the fact the M. tb PpiB bears high degree of similarity in amino acid sequence of S. mutans RopA proteins, it was speculated that M. tb PpiB may be involved in biofilm formation. The homologs of M. tb PpiB are also present in several well-known pathogenic bacteria such as Staphylococcus aureus, Staphylococcus epidermidis, Staphylococcus intermedius, Streptococcus mutans, Staphylococcus saprophyticus, Streptococcus constellatus, Pseudomonas aeruginosa that are known to make biofilm.

### Experiment 8

In Vitro Physical Interaction of M. Tb PpiB with Cyclosporine A and Acarbose

The in vitro physical interaction of M. tb PpiB with cyclosporine A and acarbose was studied using surface plasma resonance (SPR) technique using Autolab ESPRIT analyzer. The experiments were performed according to manufacturer instructions. Briefly, M. tb PpiB was diluted in PBS up to a concentration of 100 μg/ml and passed through a CM5 sensor chip for immobilization. For purified rPpiB, M. tb PpiB was cloned in pGEX-6p-1 vector and expressed in E. coli BL21(DE3) rosetta strain for protein purification, as described previously (Pandey et al., 2016). During association phase (300 seconds) cyclosporine A (10, 100, 250, 500 ug/ml) or acarbose (up to 100004) was diluted to different concentration in running buffer (PBS) and were allowed to pass over the immobilized PpiB. During dissociation phase (150 seconds) PBS was applied to sensor chip and the sensor chip was regenerated between each binding experiment with NaOH.

cyclosporine A binds to cyclophilins and inhibits its activity (Mitra et al 2006). Results of SPR analysis show that cyclosporine A (FIG. 18A) or acarbose (FIG. 18B) interacts with M. tb PpiB in a dose dependent manner and bind with high affinity. Table 3 shows Docking parameters of cyclosporine A in complex with mycobacterial cyclophilin PpiB. The binding energy value of cyclosporine A with PpiB as −5.2 Kcal/mol, indicative of the high affinity binding of PpiB with cyclosporine A.

TABLE 3

| | | | Binding Affinity | |
|---|---|---|---|---|
| S. No. | Protein | Inhibitor | (Kcal/mol) | Potential Energy |
| 1. | PpiB | cyclosporine A | −5.2 | −4.5299728e+05 |

These results suggest that cyclosporine A or acarbose, by virtue of their ability to bind to PpiB, could modulate the activity of PpiB. It was therefore speculated that modulation of PpiB activity upon binding with cyclosporine A or acarbose could affect biofilm formation per se, that in turn could enhance penetrance of drugs across biofilms, thereby lowering the MIC values of drugs and also survival of the bacteria.

### ADVANTAGES OF THE PRESENT DISCLOSURE

The present disclosure provides a medicament comprising a drug selected from a group consisting of acarboase, cyclosporine A, its pharmaceutically acceptable salts thereof, and combinations thereof, for use in treatment of diseases caused by biofilm forming microorganisms. The examples provided clearly depict that the composition of the present disclosure proves efficient enough to bind to PpiB and thus modulates its activity. Thus, the present disclosure provides an answer to problems posed by biofilm forming bacteria.

I/We claim:

1. A medicament comprising a drug selected from a group consisting of acarbose, cyclosporine A, its pharmaceutically acceptable salts thereof, and combinations thereof, for use in treatment of diseases caused by biofilm forming microorganisms.

2. The medicament as claimed in claim 1, wherein the drug is cyclosporine A for use in treatment of diseases caused by biofilm forming microorganisms.

3. The medicament as claimed in claim 1, wherein the drug is acarbose for use in treatment of diseases caused by biofilm forming microorganisms.

4. The medicament as claimed in any one of the claims 1-3, further comprising at least one compound for treatment of diseases caused by biofilm forming organisms.

5. The medicament as claimed in any one of the claims 1-4, wherein the biofilm forming microorganisms is selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

6. The medicament as claimed in any one of the claims 1-5, wherein the biofilm forming microorganism is M. tuberculosis.

7. The medicament as claimed in any one of the claims 1-4, wherein said medicament is used in the treatment of diseases selected from a group consisting of tuberculosis, dental caries, and periodontitis.

8. The medicament as claimed in claim 7, wherein said medicament is used in treatment of tuberculosis.

9. The medicament as claimed in claim 7, wherein said medicament is used in treatment of dental caries.

10. The medicament as claimed in any one of the claims 1-4, wherein the drug is a pharmaceutically acceptable salt of acarbose.

11. The medicament as claimed in any of the claims 1-4, wherein the drug is a pharmaceutically acceptable salt of cyclosporine A.

12. The medicament as claimed in claim 4, wherein said compound is selected from a group consisting of isoniazid, rifampicin, pyrazinamide, ethambutol, streptomycin, Fe₃O₄ nanocatalyst, clarithromycine, DNase, and pharmaceutically acceptable salts thereof.

13. The medicament as claimed in claim 4, wherein said compound is administered, before, concurrently, or after administration of the drug selected from a group consisting of acarbose, cyclosporine A, and pharmaceutically acceptable salts thereof.

14. The medicament as claimed in claim 13, wherein said administration of said compound, and the drug selected from a group consisting of acarbose, and cyclosporine A, can be in the form of a single dose or multiple doses.

15. A method of treatment of diseases caused by biofilm forming microorganisms, said method comprising:
(a)—obtaining a drug selected from a group consisting of acarbose and its pharmaceutically acceptable salts; and
(b) administering a therapeutically relevant amount of the drug.

16. A method of treatment of diseases caused by biofilm forming microorganisms, said method comprising:
(a) obtaining a drug selected from a group consisting of cyclosporine A and its pharmaceutically acceptable salts; and
(b) administering a therapeutically relevant amount of the drug.

17. A method of treatment of diseases caused by biofilm forming microorganisms, said method comprising:
(a) obtaining a drug selected from a group consisting of acarbose, cyclosporine A, its pharmaceutically acceptable salts thereof, and combinations thereof; and
(b) administering a therapeutically relevant amount of the drug to a subject for treatment of diseases caused by biofilm forming microorganisms.

18. The method as claimed in any one of the claims 15-17, wherein said method further comprises the step of obtaining and administering at least one more compound for treatment of the disease caused by biofilm forming microorganisms.

19. The method as claimed in any one of the claims 15-18, wherein the biofilm forming microorganisms is selected from a group consisting of Mycobacterium smegmatis, Mycobacterium tuberculosis, multidrug resistant M. tuberculosis, extensively drug resistant M. tuberculosis, totally drug resistant variant of M. tuberculosis, Staphylococcus aureus, Staphylococcus epidermidis, Staphylococcus intermedius, Streptococcus mutans, Staphylococcus saprophyticus, Streptococcus constellatus, Pseudomonas aeruginosa, and combinations thereof.

20. The method as claimed in any one of the claims 15-18, wherein the biofilm forming microorganism is M. tuberculosis.

21. The method as claimed in any one of the claims 15-18, wherein said method is used for treatment of diseases selected from a group consisting of tuberculosis, dental caries, and periodontitis.

22. The method as claimed in any one of the claims 15-18, wherein said method is used for treatment of tuberculosis.

23. The method as claimed in any one of the claims 15-18, wherein said method is used for treatment of dental caries.

24. The method as claimed in claim 18, wherein said compound is selected from a group consisting of isoniazid, rifampicin, pyrazinamide, ethambutol, streptomycin, Fe₃O₄ nanocatalyst, clarithromycine, DNase, and pharmaceutically acceptable salts.

25. The method as claimed in any one of the claims 15-18, wherein said compound is administered before, concurrently or after administration of the drug selected from a group consisting of acarbose, and cyclosporine A.

26. The method as claimed in claim 25, wherein administration of the drug selected from a group consisting of acarbose, and cyclosporine A can be in the form of a single dose or multiple doses.
